(19) United States
(12) Patent Application Publication
(10) Pub. No.: US 2010/0048633 A1
(43) Pub. Date: Feb. 25, 2010

(54) Piperidine-4-acrylamides

(76) Inventors: Philipp Lehr, Vienna (AT); Klaus Weigand, Vienna (AT); Anny-Odile Colson, Horsham (GB)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/523,130

(22) PCT Filed: Jan. 29, 2008
(86) PCT No.: PCT/EP2008/051011

§ 371 Date: Jul. 14, 2009

(30) Foreign Application Priority Data
Jan. 31, 2007 (EP) 07101466.6

(51) Int. Cl. A61K 31/445; C07D 211/34; C07D 401/12; A61K 31/454; A61P 29/00

(52) U.S. Cl. 514/326; 546/234; 546/210; 514/277; 514/331

(57) ABSTRACT

A compound of formula (I), wherein substituents are as given above, useful in the treatment of a disease mediated by the action of CCR3.

The present invention relates to piperidine-4-acrylamides, e.g. the compounds of given formula and their use as pharmaceuticals.

In one aspect the present invention provides a compound of formula

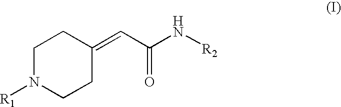

wherein
R₁ and R₂ independently are (C₆₋₁₈)aryl or (C₆₋₁₈)aryl(C₁₋₆)alkyl one or morefold substituted by (C₁₋₆)alkyl, (C₁₋₆)alkoxy, halo(C₁₋₆)alkyl, halogen or unsubstituted or substituted heterocyclyl having 5 or 6 ring members and 1 to 4 heteroatoms selected from N, O, S.

In another aspect the present invention provides a compound of formula (I), wherein
R₁ and R₂ independently are phenyl or phenyl(C₁₋₄)alkyl one or morefold substituted by (C₁₋₄)alkyl, halogen or unsubstituted or substituted heterocyclyl having 5 ring members and 1 to 4 heteroatoms selected from N, O, S.

In another aspect the present invention provides a compound of formula (I), wherein
R₁ is phenyl, benzyl or phenethyl, one or twofold substituted by methyl or fluoro,
R₂ is phenyl, phenyl-methyl(benzyl) or phenyl-ethyl(phenethyl) one or twofold substituted by methyl, fluoro or 1-methyl-1 H-tetrazol-5-yl.

If not otherwise defined herein
Alkyl includes (C₁₋₆)alkyl, e.g. (C₁₋₄)alkyl, such as e.g. methyl;
Alkoxy includes (C₁₋₆)alkoxy, e.g. (C₁₋₄)alkoxy, such as e.g. methoxy;
Aryl includes (C₆₋₁₈)aryl, e.g. phenyl, optionally anellated with (C₆₋₁₈)aryl, e.g. phenyl;
Aryl-alkyl includes (C₆₋₁₈)aryl(C₁₋₆)alkyl such as e.g. phenyl(C₁₋₆)alkyl, e.g. phenyl-methyl(benzyl) or phenyl-ethyl(phenetyl);
Heterocyclyl includes a 5 or 6 membered aromatic or non-aromatic ring system having 1 to 4 heteroatoms selected from N, O, S; e.g. N, such as e.g. tetrazolyl, preferably 1-methyl-1H-tetrazol-5-yl;
Halogen includes fluoro, chloro, bromo, e.g. fluoro;
Haloalkyl includes halo(C₁₋₆)alkyl, e.g. halo(C₁₋₄)alkyl, wherein halo is one or more halogen, preferably trifluoromethyl;

Any group may be unsubstituted or substituted, e.g. substituted by groups as conventional in organic chemistry, e.g. including groups selected from halogen, haloalkyl, alkylcarbonyloxy, alkoxy, hydroxy, amino, alkylcarbonylamino, aminoalkylcarbonylamino, hydroxyalkylamino, aminoalkylamino, alkylamino, dialkylamino, heterocyclyl having 5 or 6 ring members and 1 to 4 heteroatoms selected from N,O,S; (C₁₋₄)alkylheterocyclyl, wherein heterocyclyl having 5 or 6 ring members and 1 to 4 heteroatoms selected from N,O,S; hydroxy(C₁₋₄)alkylheterocyclyl, wherein heterocyclyl having 5 or 6 ring members and 1 to 4 heteroatoms selected from N,O,S; carboxyl, (C₁₋₄)alkylcarbonyloxy, amino(C₁₋₄)-alkylcarbonyloxy.

In a compound of formula (I) each single defined substitutent may be a preferred substituent, e.g. independently of each other substitutent defined.

In a compound of formula (I) preferably R₁ is phenyl, benzyl or phenethyl, one or twofold substituted by methyl or fluoro, and R₂ is as defined above.

In a compound of formula (I) preferably R₂ is phenyl, benzyl or phenethyl, one or twofold substituted by methyl, fluoro or 1-methyl-tetrazol-5-yl, and R₁ is as defined above.

In another aspect the present invention provides a compound selected from the group consisting of
N-(3,4-Difluoro-phenyl)-2-[1-(4-fluoro-benzyl)-piperidin-4-ylidene]-acetamide
N-(3,4-Difluoro-phenyl)-2-[1-(3-methyl-4-fluoro-benzyl)-piperidin-4-ylidene]-acetamide
N-(3,4-Difluoro-phenyl)-2-[1-(3-fluoro-4-methyl-benzyl)-piperidin-4-ylidene]-acetamide
N-(3,4-Difluoro-phenyl)-2-[1-(3,4-dimethyl-benzyl)-piperidin-4-ylidene]-acetamide
N-(3,4-Difluoro-benzyl)-2-[1-(4-fluoro-benzyl)-piperidin-4-ylidene]-acetamide
N-(3,4-Difluoro-benzyl)-2-[1-(3-methyl-4-fluoro-benzyl)-piperidin-4-ylidene]-acetamide
N-(3,4-Difluoro-benzyl)-2-[1-(3-fluoro-4-methyl -benzyl)-piperidin-4-ylidene]-acetamide
N-(3,4-Difluoro-benzyl)-2-[1-(3,4-dimethyl -benzyl)-piperidin-4-ylidene]-acetamide
2-[1-(4-Fluoro-3-methyl-benzyl)-piperidin-4-ylidene]-N-[2-(4-fluoro-phenyl)-ethyl]-acetamide
2-[1-(3-Fluoro-4-methyl-benzyl)-piperidin-4-ylidene]-N-[2-(4-fluoro-phenyl)-ethyl]-acetamide
2-[1-(4-Fluoro-3-methyl-benzyl)-piperidin-4-ylidene]-N-[2-(4-fluoro-phenyl)-ethyl]-acetamide
2-[1-(3,4-Dimethyl-benzyl)-piperidin-4-ylidene]-N-[2-(4-fluoro-phenyl)-ethyl]-acetamide
2-[1-(4-Fluoro-benzyl)-piperidin-4-ylidene]-N-[3-(1-methyl-1H-tetrazol-5-yl)-phenyl]-acetamide
2-[1-(4-Fluoro-3-methyl-benzyl)-piperidin-4-ylidene]-N-[3-(1-methyl-1H-tetrazol-5-yl)-phenyl]-acetamide
2-[1-(3-Fluoro-4-methyl-benzyl)-piperidin-4-ylidene]-N-[3-(1-methyl-1H-tetrazol-5-yl)-phenyl]-acetamide
2-[1-(3,4-Dimethyl-benzyl)-piperidin-4-ylidene]-N-[3-(1-methyl-1H-tetrazol-5-yl)-phenyl]-acetamide
N-(3,4-Difluoro-benzyl)-2-{1-[2-(4-fluoro-phenyl)-ethyl]-piperidin-4-ylidene}-acetamide
N-(3,4-Difluoro-phenyl)-2-{1-[2-(4-fluoro-phenyl)-ethyl]-piperidin-4-ylidene}-acetamide
N-[2-(4-Fluoro-phenyl)-ethyl]-2-{1-[2-(4-fluoro-phenyl)-ethyl]-piperidin-4-ylidene}-acetamide
2-{1-[2-(4-Fluoro-phenyl)-ethyl]-piperidin-4-ylidene}-N-[3-(1-methyl-1H-tetrazol-5-yl)-phenyl]-acetamide

The nomenclature used for the compounds mentioned above is according to the nomenclature obtained when using the Software “ISIS draw, Version 2.5”.

Compounds used by or provided by the present invention are hereinafter designated as “compound(s) of (according to) the present invention”. A compound of the present invention includes a compound in any form, e.g. in free form, in the form of a salt, in the form of a solvate and in the form of a salt and a solvate.

In another aspect the present invention provides a compound of the present invention in the form of a salt.

Such salts include preferably pharmaceutically acceptable salts, although pharmaceutically unacceptable salts are included, e.g. for preparation/isolation/purification purposes.

A salt of a compound of the present invention includes a metal salt or an acid addition salt. Metal salts include for example alkali or earth alkali salts; acid addition salts include salts of a compound of formula (I) with an acid, e.g. hydrogen fumaric acid, fumaric acid, naphthalin-1,5-sulphonic acid, hydrochloric acid, deuterochloric acid; preferably hydrochloric acid.

A compound of the present invention in free form may be converted into a corresponding compound in the form of a salt; and vice versa. A compound of the present invention in free form or in the form of a salt and in the form of a solvate may be converted into a corresponding compound in free form or in the form of a salt in non-solvated form; and vice versa.

A compound of the present invention may exist in the form of pure isomers or mixtures thereof; e.g. optical isomers, diastereoisomers, cis/trans isomers. A compound of the present invention may e.g. contain asymmetric carbon atoms and may thus exist in the form of enantiomers or diastereoisomers and mixtures thereof, e.g. racemates. Any asymmetric carbon atom may be present in the (R)-, (S)- or (R,S)-configuration, preferably in the (R)- or (S)-configuration.

Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of formula (I), where tautomers can exist.

In another aspect the present invention provides a process for the production of a compound of formula (I) comprising the steps

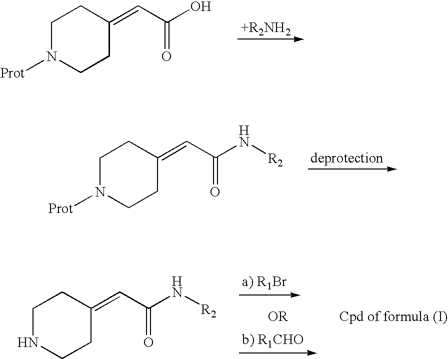

wherein Prot is a protecting group, e.g. Boc, and isolating a compound of formula (I) obtained from the reaction mixture.

If route a) is used the reaction is carried out e.g. in the presence of Et₃N, KJ, CH₃CN, at appropriate temperatures and for the appropriate time.

If route b) is used the reaction is carried out e.g. in the presence of NaBH(OAc)₃, THF, DIEA at appropriate temperatures and for the appropriate time.

In an intermediate (starting materials), functional groups, if present, optionally may be in protected form or in the form of a salt, if a salt-forming group is present. Protecting groups, optionally present, may be removed at an appropriate stage, e.g. according, e.g. analogously, to a method as conventional.

A compound of formula (I) thus obtained may be converted into another compound of formula (I), e.g. or a compound of formula (I) obtained in free form may be converted into a salt of a compound of formula (I) and vice versa.

The above reaction may be carried out as appropriate, e.g. analogously to a method as conventional.

Intermediates (starting materials) are known or may be prepared according, e.g. analogously, to a method as conventional or as described herein.

Any compound described herein, e.g. a compound of the present invention and intermediates of formula I_{A} or of formula I_{B} may be prepared as appropriate, e.g. according, e.g. analogously, to a method as conventional, e.g. or as specified herein.

The compounds of the present invention, e.g. including a compound of formula (I), exhibit pharmacological activity and are therefore useful as pharmaceuticals. E.g., the compounds of formula (I) are useful for the manufacture of a medicament, e.g. for the treatment of diseases mediated by CCR3.

The compounds of the present invention act as CCR3 receptor antagonists, thereby inhibiting the infiltration and activation of inflammatory cells, particularly eosinophils, and inhibiting allergic response. The inhibitory properties of the compounds of the present invention can be demonstrated in the following assay:

In this assay the effect of the compounds of the present invention on the binding of human eotaxin to human CCR3 is determined. Recombinant cells expressing human CCR3 are captured by wheatgerm agglutinin (WGA) polyvinyltoluidene (PVT) SPA beads (available from Amersham), through a specific interaction between the WGA and carbohydrate residues of glycoproteins on the surface of the cells. [¹²⁵I]-human eotaxin (available from Amersham) binds specifically to CCR3 receptors bringing the [¹²⁵I]-human eotaxin in close proximity to the SPA beads. Emitted â-particles from the [¹²⁵I]-human eotaxin excite, by its proximity, the fluorophore in the beads and produce light. Free [¹²⁵I]-human eotaxin in solution is not in close proximity to the scintillant and hence does not produce light. The scintillation count is therefore a measure of the extent to which the test compound inhibits binding of the eotaxin to the CCR3.

Preparation of Assay Buffer 5.96 g HEPES and 7.0 g sodium chloride are dissolved in distilled H₂O and 1 M aq. CaCl₂ (1 ml) and 1 M aq. MgCl₂ (5 ml) are added. The pH is adjusted to 7.6 with NaOH and the solution made to a final volume of 1 L using distilled H₂O. 5 g of bovine serum albumin and 0.1 g NaN₃ are dissolved in the solution and the resulting buffer stored at 4° C. A Complete™ protease inhibitor cocktail tablet (available from Boehringer) is added per 50 ml of the buffer on the day of use.

Preparation of Homogenisation Buffer: Tris-base (2.42 g) is dissolved in distilled H₂O, the pH of the solution is adjusted to 7.6 with HCl and the solution obtained is diluted with distilled H₂O to a final volume of 1 l. The resulting buffer is stored at 4° C. A Complete™ protease inhibitor cocktail tablet is added per 50 ml of the buffer on the day of use.

Preparation of membranes: Confluent rat basophil leukaemia (RBL-2H3) cells stably expressing CCR3 are removed from tissue culture flasks using enzyme-free cell dissociation buffer and resuspended in phosphate-buffered saline. The cells are centrifuged (800 g, 5 minutes), the pellet obtained is resuspended in ice-cold homogenisation buffer using 1 ml homogenisation buffer per gram of cells and incubated on ice for 30 minutes. The cells are homogenised on ice with 10 strokes in a glass mortar and pestle. The homogenate is centrifuged (800 g, 5 minutes, 4° C.), the supernatant obtained is centrifuged (48,000 g, 30 minutes, 4° C.) and the pellet obtained is redissolved in Homogenisation Buffer containing 10% (v/v) glycerol. The protein content of the membrane preparation is estimated by the method of Bradford (Anal. Biochem. (1976) 72:248) and aliquots are snap frozen and stored at −80° C.

The assay is performed in a final volume of 250 μl per well of an Optiplate™ microplate (ex Canberra Packard). 50 μl of solutions of a test compound in Assay Buffer containing 5% DMSO (concentrations from 0.01 nM to 10 μM) are added to selected wells of the microplate. To determine total binding, 50 μl of the Assay Buffer containing 5% DMSO is added to other selected wells. To determine non-specific binding, 50 μl of 100 nM human eotaxin (ex R&D Systems) in Assay Buffer containing 5% DMSO is added to further selected wells. 50 μl of [¹²⁵I]-Human eotaxin (ex Amersham) in Assay Buffer containing 5% DMSO at a concentration of 250 pM (to give a final concentration of 50 pM per well), 50 μl of WGA-PVT SPA beads in Assay Buffer (to give a final concentration of 1.0 mg beads per well) and 100 μl of the membrane preparation at a concentration of 100 μg protein in Assay Buffer (to give a final concentration of 10 μg protein per well) are added to all wells. The plate is then incubated for 4 hours at room temperature. The plate is sealed using TopSeal-S™ sealing tape (ex Canberra Packard) according to the manufacturer's instructions. The resulting scintillations are counted using a Canberra Packard TopCount™ scintillation counter, each well being counted for 1 minute. The concentration of test compound at which 50% inhibition occurs (IC₅₀) is determined from concentration-inhibition curves in a conventional manner.

The compounds of the Examples herein below generally have IC₅₀ values below 1 μM in the above assay, e.g. a compound of example 17 has an IC₅₀ value of about 0.2 μM.

Most of the compounds of the Examples exhibit selectivity for inhibition of CCR3 binding relative to inhibition of binding of the alpha-1 adrenergic receptor.

The inhibitory properties of the compounds of the present invention on binding of the alpha-1 adrenergic receptor can be determined in the following assay:

Cerebral cortices from male Sprague-Dawley rats (175-200 g) are dissected and homogenised in 10 volumes of ice cold 0.32 M sucrose (containing 1 mM MgCl₂ dihydrate and 1 mM K₂HPO₄) with a glass/Teflon homogeniser. The membranes are centrifuged at 1000×g for 15 minutes, the pellet discarded and the centrifugation repeated. The supernatants are pooled and centrifuged at 18,000×g for 15 minutes. The pellet is osmotically shocked in 10 volumes of H₂O and kept on ice for 30 minutes. The suspension is centrifuged at 39,000×g for 20 minutes, resuspended in Krebs-Henseleit buffer pH 7.4 (1.17 mM MgSO₄ anhydrous, 4.69 mM KCl, 0.7 mM K₂HPO₄ anhydrous, 0.11 M NaCl, 11 mM D-glucose and 25 mM NaHCO₃) containing 20 mM Tris, and kept for 2 days at −20° C. The membranes are thawed at 20-23° C., washed three times with Krebs-Henseleit buffer by centrifugation at 18,000×g for 15 minutes, left overnight at 4° C. and washed again 3 times. The final pellet is resuspended with a glass/Teflon homogeniser in 125 ml/100 membranes in the same buffer. A sample is taken to determine the protein concentration (using the Bradford Assay with gamma globulin as the standard) and the remainder aliquoted and stored at −80° C.

The resulting membranes are subjected to a radioligand binding assay. The assay is conducted in triplicate using 96 well plates containing [¹²⁵I]-HEAT (Amersham) (40 pM, Kd: 58.9±18.7 pM), unlabelled test compound and membrane (57.1 μg/ml) to yield a final volume of 250 μl (assay buffer containing 50 mM Tris-base and 0.9% (w/v) NaCl, pH 7.4). The plates are incubated at 37° C. for 60 minutes, after which rapid vacuum filtration over Whatman™ GF/C 96 well filter plates is carried out. Each plate is then washed three times with 10 ml of ice cold assay buffer using a Brandel Cell harvester (Gaithersburg, Md.). Following drying of the plates for 3 h. at 50° C., 40 μl of Microscint 20 is added to each well, the plates incubated at room temperature for a further 20 minutes and the retained radioactivity quantified in a Packard TopCount NXT™ scintillation counter.

Stock solutions of test compounds are dissolved initially in 100% DMSO and diluted with assay buffer to the required concentrations to yield 1% (v/v) DMSO. The concentration of test compound at which 50% inhibition occurs (IC₅₀) is determined from concentration-inhibition curves in a conventional manner.

With regard to their inhibition of binding of CCR3, the compounds of the present invention are useful in the treatment of conditions mediated by CCR3, particularly inflammatory or allergic conditions. Treatment in accordance with the present invention may be symptomatic or prophylactic.

Accordingly, compounds of the present invention are useful in the treatment of inflammatory or obstructive airways diseases, resulting, for example, in reduction of tissue damage, bronchial hyper-reactivity, remodelling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial or viral infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. cortico-steroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Other inflammatory or obstructive airways diseases and conditions to which the present invention is applicable include acute lung injury (ALI), acute/adult respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The present invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Having regard to their anti-inflammatory activity, in particular in relation to inhibition of eosinophil activation, compounds of the present invention are also useful in the treatment of eosinophil related disorders, e.g. eosinophilia, in particular eosinophil related disorders of the airways (e.g. involving morbid eosinophilic infiltration of pulmonary tissues) including hyper-eosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

The compounds of the present invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, and other inflammatory or allergic conditions of the skin.

The compounds of the present invention may also be used for the treatment of other diseases or conditions, in particular diseases or conditions having an inflammatory component, for example, treatment of diseases and conditions of the eye such as conjunctivitis, keratoconjunctivitis sicca, and vernal conjunctivitis, diseases affecting the nose including allergic rhinitis, e.g. atrophic, chronic, or seasonal rhinitis, inflammatory conditions of the gastrointestinal tract, for example inflammatory bowel disease such as ulcerative colitis and Crohn's disease, diseases of the bone and joints including rheumatoid arthritis, psoriatic arthritis, ankylosing spondylitis and systemic sclerosis, and other diseases such as cystic fibrosis, pulmonary hypertension, atherosclerosis, multiple sclerosis, diabetes (type I), myasthenia gravis, hyper IgE syndrome and acute and chronic allograft rejection, e.g. following transplantation of heart, kidney, liver, lung or bone marrow.

The effectiveness of a compound of the present invention in inhibiting inflammatory conditions, for example in inflammatory airways diseases, may be demonstrated in an animal model, e.g. a mouse or rat model, of airways inflammation or other inflammatory conditions, for example as described by Szarka et al, J. Immunol. Methods (1997) 202:49-57; Renzi et al, Am. Rev. Respir. Dis. (1993) 148:932-939; Tsuyuki et al., J. Clin. Invest. (1995) 96:2924-2931; and Cernadas et al (1999) Am. J. Respir. Cell Mol. Biol. 20:1-8.

The compounds of the present invention are also useful as co-therapeutic compounds for use in combination with other drug substances such as anti-inflammatory, bronchodilatory, antihistamine or anti-tussive drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the present invention may be mixed with the other drug substance in a fixed pharmaceutical composition or it may be administered separately, before, simultaneously with or after the other drug substance.

Such anti-inflammatory drugs include steroids, in particular glucocorticosteroids such as budesonide, beclamethasone, fluticasone, ciclesonide or mometasone, or steroids described in WO 02/88167, WO 02/12266, WO 02/100879, WO 04/039827 or WO 02/00679, especially those of Examples 3, 11, 14, 17, 19, 26, 34, 37, 39, 51, 60, 67, 72, 73, 90, 99 and 101; LTB4 antagonists such as those described in U.S. Pat. No. 5,451,700, also LY2931 11, CGS025019C, CP-1 95543, SC-53228, BIIL 284, ONO 4057 and SB 209247; LTD4 antagonists such as montelukast and zafirlukast; Dopamine receptor agonists such as cabergoline, bromocriptine, ropinirole and 4-hydroxy-7-[2-[[2-[[3-(2-phenylethoxy)propyl]sulfonyl]ethyl]-amino]ethyl]-2(3H)-benzothiazolone and pharmaceutically acceptable salts thereof (the hydrochloride being Viozane®-AstraZeneca); PDE4 inhibitors such as cilomilast (Ariflo® GSK), Roflumilast (Byk Gulden), V-1 1294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeIClD(TM) CC-10004 (Celgene), VM554/UM565 (Vernalis), T-440 (Tanabe), KW-4490 (Kyowa Hakko Kogyo), WO 92/19594, WO 93/19749, WO 93/19750, WO 93/19751, WO 99/16766, WO 01/13953, WO 03/104204, WO 03/104205, WO 04/000814, WO 04/000839 and WO 04/005258, WO 04018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945 and WO 04/045607, WO 04/037805 as well as those described in WO 98/18796 and WO 03/39544; A2a agonists such as those described in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618, WO 04/046083; and A2b antagonists such as those described in WO 02/42298.

Such bronchodilatory drugs include anticholinergic or antimuscarinic agents, in particular ipratropium bromide, oxitropium bromide, tiotropium bromide, CHF 4226 (Chiesi) and glycopyrrolate, but also those described in WO 01/04118, WO 02/51841, WO 02/53564, WO 03/00840, WO 03/87094, WO 04/05285, WO 02/00652, WO 03/53966, EP 424021, U.S. Pat. No. 5,171,744, U.S. Pat. No. 3,714,357, U.S. Pat. No. 5,171,744, WO 03/33495 and WO 04/018422; and beta (β)-2-adrenoceptor agonists such as albuterol (salbutamol), metaproterenol, terbutaline, salmeterol, fenoterol, procaterol, and especially, formoterol and pharmaceutically acceptable salts thereof, and compounds (in free or salt or solvate form) of formula (I) of WO 00/75114, which document is incorporated herein by reference, preferably compounds of the Examples thereof, especially a compound of formula

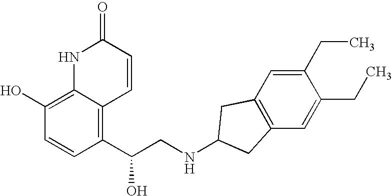

and pharmaceutically acceptable salts thereof, as well as compounds (in free or salt or solvate form) of formula (I) of WO 04/16601. Further suitable,8-2-adrenoreceptor agonists include compounds such as those described in JP 05025045, US 2002/0055651, WO 93/18007, WO 99/64035, WO 01/42193, WO 01/83462, WO 02/066422, WO 02/070490, WO 02/076933, WO 03/24439, WO 03/72539, WO 03/42160, WO 03/91204, WO 03/42164, WO 03/99764, WO 04/11416, WO 04/16578, WO 04/22547, WO 04/32921, WO 04/33412, WO 04/37773, WO 04/37807, WO 04/39762, WO 04/39766, WO 04/45618 and WO 04/46083.

Such co-therapeutic antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in JP 2004107299, WO 03/99807 and WO 04/26841.

Combinations of compounds of the present invention and one or more steroids, beta-2 agonists, PDE4 inhibitors or LTD4 antagonists may be used, for example, in the treatment of COPD or, particularly, asthma. Combinations of compounds of the present invention and anticholinergic or antimuscarinic agents, PDE4 inhibitors, dopamine receptor agonists or LTB4 antagonists may be used, for example, in the treatment of asthma or, particularly, COPD.

Other useful combinations of compounds of the present invention with anti-inflammatory drugs are those with other antagonists of chemokine receptors, e.g. CCR1, CCR2, CCR4, CCR5, CCR6, CCR7, CCR8, CCR9 and CCR10, CXCR1, CXCR2, CXCR3, CXCR4, CXCR5, particularly CCR5 antagonists such as Schering-Plough antagonists SC-351125, SCH-55700 and SCH-D, Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770), CCR5 antagonists described in U.S. Pat. No. 6,166,037 (particularly claims 18 and 19), WO 00/66558 (particularly claim 8), and WO 00/66559 (particularly claim 9), WO 04/018425 and WO 04/026873.

In accordance with the foregoing, the present invention also provides a method for the treatment of a condition mediated by CCR3, for example an inflammatory or allergic condition, particularly an inflammatory or obstructive airways disease, which comprises administering to a subject, particularly a human subject, in need thereof an effective amount of a compound of formula (I) in a free or pharmaceutically acceptable salt form as hereinbefore described.

In another aspect the present invention provides the use of a compound of formula (I), in free or pharmaceutically acceptable salt form, as hereinbefore described for the manufacture of a medicament for the treatment of a condition mediated by CCR3, e.g. an inflammatory or allergic condition, particularly an inflammatory or obstructive airways disease.

The compounds of the present invention may be administered by any appropriate route, e.g. orally, for example in the form of a tablet or capsule; parenterally, for example intravenously; by inhalation, for example in the treatment of inflammatory or obstructive airways disease; intranasally, for example in the treatment of allergic rhinitis; topically to the skin, e.g. in the treatment of atopic dermatitis; or rectally, e.g. in the treatment of inflammatory bowel disease.

In a further aspect, the present invention also provides a pharmaceutical composition comprising as active ingredient a compound of formula (I) in free or pharmaceutically acceptable salt form, optionally together with a pharmaceutically acceptable diluent or carrier therefor. The composition may contain a co-therapeutic agent such as an anti-inflammatory bronchodilatory or antihistamine drug as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g. patches. Compositions for inhalation may comprise aerosol or other atomizable formulations or dry powder formulations.

When the composition comprises an aerosol formulation, it preferably contains, for example, a hydro-fluoro-alkane (HFA) propellant such as HFA134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it preferably contains, for example, the compound of formula (I) having a particle diameter up to 10 microns, optionally together with a diluent or carrier, such as lactose, of the desired particle size distribution and a compound that helps to protect against product performance deterioration due to moisture e.g. magnesium stearate. When the composition comprises a nebulised formulation, it preferably contains, for example, the compound of formula (I) either dissolved, or suspended, in a vehicle containing H₂O, a co-solvent such as EtOH or propylene glycol and a stabiliser, which may be a surfactant.

The present invention includes (A) a compound of the present invention in inhalable form, e.g. in an aerosol or other atomisable composition or in inhalable particulate, e.g. micronised form, (B) an inhalable medicament comprising a compound of the present invention in inhalable form; (C) a pharmaceutical product comprising such a compound of the present invention in inhalable form in association with an inhalation device; and (D) an inhalation device containing a compound of the present invention in inhalable form.

Dosages of compounds of the present invention employed in practising the present invention will of course vary depending, for example, on the particular condition to be treated, the effect desired and the mode of administration. In general, suitable daily dosages for administration by inhalation are of the order of 0.01 to 30 mg/kg while for oral administration suitable daily doses are of the order of 0.01 to 100 mg/kg.

In the following Examples all temperatures are given in ° C.

The following abbreviations are used:
aq. aqueous
DIEA diisopropylethylamine
DMAP N,N-dimethyl-4-aminopyridin
EtOAc ethyl acetate
NMM N-methyl-morpholine
PPA 1-propanephosphonic acid cyclic anhydride
RT room temperature
sat. saturated
THF tetrahydrofuran

### EXAMPLES

### Example 1

N-(3,4-difluoro-benzyl)-2-piperidin-4-ylidene-acetamide hydrochloride is added to a solution of 4-fluoro-benzaldehyde, NaBH(OAc)₃ and DIEA in THF. The mixture obtained is stirred for 18 hours at ambient temperature and concentrated in vacuo. The residue obtained is taken up in EtOAc, sat. aq. NaHCO₃-solution is added and the layers obtained are separated. The aq. phase obtained is extracted 2× with EtOAc and the combined organic extracts obtained are washed with brine, dried over Na₂SO₄ and concentrated. The residue obtained is chromatographed on silica gel (eluent:EtOAc). N-(3,4-Difluoro-benzyl)-2-[1-(4-fluoro-benzyl)-piperidin-4-ylidene]-acetamide is obtained,

mp. 83-85° C.

The following compounds of formula (I) are prepared analogously as described in Example 1:

| | | | MH+ or |
|---|---|---|---|
| Example | R₁ | R₂ | mp. when indicated |
| 2 | 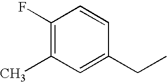 | 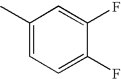 | 79-82 (mp.) |
| 3 | 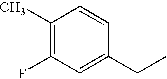 | 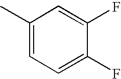 | 133-136 (mp.) |
| 4 | 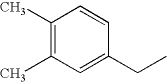 | 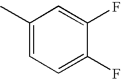 | 165-168 (mp.) |
| 5 | 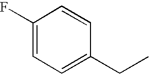 | 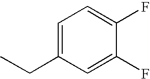 | 83-85 (mp.) |
| 6 | 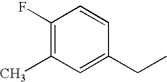 | 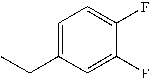 | 411 |
| 7 | 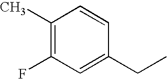 | 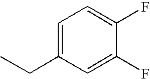 | 389 |
| 8 | 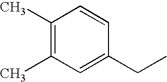 | 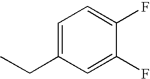 | 385 |
| 9 | 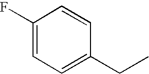 | 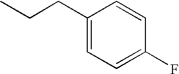 | 371 |
| 10 | 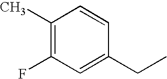 | 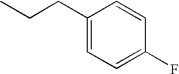 | 385 |
| 11 | 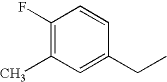 | 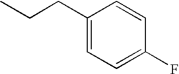 | 385 |
| 12 | 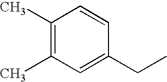 | 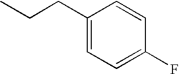 | 381 |
| 13 | 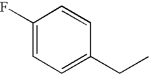 | 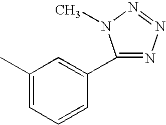 | 65-68 (mp.) |
| 14 | 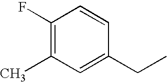 | 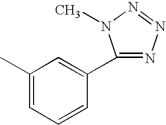 | 64-67 (mp.) |
| 15 | 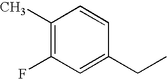 | 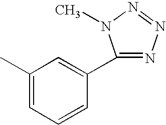 | 55-57 (mp.) |
| 16 | 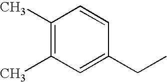 | 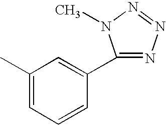 | 74-76 (mp.) |

### Example 17

### N-(3,4-Difluoro-benzyl)-2-{1-[2-(4-fluoro-phenyl)-ethyl]-piperidin-4-ylidene}-acetamide

### a) 4-[(3,4-Difluoro-benzylcarbamoyl)-methylene]-piperidine-1-carboxylic acid tert.butyl ester

PPA (50% solution in DMF) is added to a solution of 4-carboxy-methylene-piperidine-1-carboxylic acid tert.butyl ester, 3,4-difluoro-benzylamine, DMAP and NMM in 20 ml CH₂Cl₂. The reaction mixture obtained is stirred for 18 hours at RT, evaporated in vacuo, and the residue obtained is taken up in EtOAc, washed with 0.1 N HCl and brine, and dried over MgSO₄. The solution obtained is concentrated in vacuo and the residue obtained is chromatographed on silica gel (eluent:cyclohexane/ethylacetate=2:1). 4-[(3,4-Difluoro-benzylcarbamoyl)-methylene]-piperidine-1-carboxylic acid tert.butyl ester is obtained.

mp. 102-105° C., MS: 389 (MNa+), 365 (M−H)

### b) N-(3,4-Difluoro-benzyl)-2-piperidin-4-ylidene-acetamide hydrochloride

2M HCl solution in diethylether is added to a solution of 4-[(3,4-difluorobenzylcarbamoyl)-methylene]-piperidine-1-carboxylic acid tert.butyl ester in CH₂Cl₂. The mixture obtained is stirred for 18 hours at RT, a precipitate obtained is filtered off, washed with diethylether and dried. N-(3,4-Difluoro-benzyl)-2-piperidin-4-ylidene-acetamide hydrochloride is obtained.

mp. 43-45° C., MS: 267 (MH+)

### c) N-(3,4-Difluoro-benzyl)-2-{1-[2-(4-fluoro-phenyl)-ethyl]-piperidin-4-ylidene}-acetamide

N-(3,4-difluoro-benzyl)-2-piperidin-4-ylidene-acetamide hydrochloride is dissolved in CH₂Cl₂ and Et₃N. 4-fluorophenethylbromide and KI are added. The mixture obtained is refluxed for 4 hours, cooled to RT, evaporated in vacuo, taken up in EtOAc, washed with sat. NaHCO₃-solution and brine, and dried over MgSO₄. The solution obtained is concentrated in vacuo and the residue obtained is chromatographed on silica gel (eluent:cyclohexane/ethylacetate=1:1). N-(3,4-Difluoro-benzyl)-2-{1-[2-(4-fluoro-phenyl)-ethyl]-piperidin-4-ylidene}-acetamide is obtained.

mp. 117-120° C., MS: 389 (MH+), 411 (MNa+), 387 (M−H)

The following compounds of formula (I) are prepared analogously as described in Example 17:

| | | | MH+ or |
|---|---|---|---|
| Example | R₁ | R₂ | mp. when indicated |
| 18 | 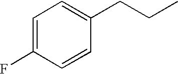 | 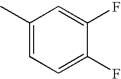 | 375 |
| 19 | 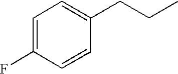 | 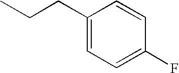 | 385 |
| 20 | 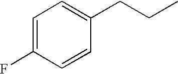 | 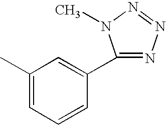 | 168-170 (mp.) |

1. A compound of formula 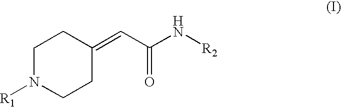
wherein
R₁ and R₂ independently are (C₆₋₁₈)aryl or (C₆₋₁₈)aryl(C₁₋₆)alkyl one or morefold substituted by
(C₁₋₆)alkyl, (C₁₋₆)alkoxy, halo(C₁₋₆)alkyl, halogen or unsubstituted or substituted heterocyclyl having 5 or 6 ring members and 1 to 4 heteroatoms selected from N, O, S.

2. A compound of formula (I) of claim 1, wherein
R₁ and R₂ independently are phenyl or phenyl(C₁₋₄)alkyl one or morefold substituted by (C₁₋₄)alkyl, halogen or unsubstituted or substituted heterocyclyl having 5 ring members and to 4 heteroatoms selected from N, O, S.

3. A compound of formula (I) of any one of claims 1, wherein
R₁ is phenyl, benzyl or phenethyl one or twofold substituted by methyl or fluoro,
R₂ is phenyl, benzyl or phenethyl one or twofold substituted by methyl, fluoro or 1-methyl-tetrazol-5-yl.

4. A compound of claim 1 in the form of a salt.

5. A compound of claim 1 for use as a pharmaceutical.

6. (canceled)

7. A pharmaceutical composition comprising a compound of claim 1 in association with at least one pharmaceutical excipient.

8. A pharmaceutical composition according to claim 7 further comprising another pharmaceutically active agent.

9. A method of treatment of a disease mediated by CCR3, which treatment comprises administering to a subject in need of such treatment an effective amount of a compound of formula (I) of claim 1.

10. A method of treatment of claim 9 wherein the disease is an inflammatory or allergic disease, particularly an inflammatory or obstructive airways disease.

11. A method of claim 9, wherein a compound of formula (I) of claim 1 is administered in combination with another pharmaceutically active agent, either simultaneously or in sequence.
